(19) United States
(12) Patent Application Publication
(10) Pub. No.: US 2025/0098980 A1
(43) Pub. Date: Mar. 27, 2025

(54) MAGNETIC RESONANCE SCANNING SYSTEM AND METHOD

(71) Applicant: GE Precision Healthcare LLC, Waukesha, WI (US)

(72) Inventors: Yanting Huo, Beijing (CN); Fan Yang, Beijing (CN); Kun Wang, Beijing (CN)

(21) Appl. No.: 18/895,047

(22) Filed: Sep. 24, 2024

(30) Foreign Application Priority Data
Sep. 26, 2023 (CN) 202311258926.6

(51) Int. Cl. A61B 5/055; A61B 34/10

(52) U.S. Cl. CPC A61B 5/055; A61B 34/10; A61B 2034/107

(57) ABSTRACT

A magnetic resonance imaging (MRI) scanning system and a method is presented. The method comprises: preconfiguring a plurality of scan configurations, the plurality of scan configurations corresponding to a plurality of scan ranges of a subject to be scanned, and each of the scan configurations comprising an implant-related MRI scan parameter; and executing a scan procedure on the subject to be scanned using at least one of the preconfigured plurality of scan configurations.

### CROSS REFERENCE TO RELATED APPLICATIONS

The present application claims priority and benefit of Chinese Patent Application No. 202311258926.6 filed on Sep. 26, 2023, which is incorporated herein by reference in its entirety.

### TECHNICAL FIELD

Embodiments of the present invention generally relate to medical imaging technology, and particularly relate to a magnetic resonance imaging (MRI) scanning system and an MRI scanning method.

### BACKGROUND

Magnetic resonance imaging technology has been widely used in the field of medical diagnostics. MRI systems usually have a main magnet, a gradient drive system, a gradient coil assembly, a radio frequency transmit chain module, a radio frequency receive chain module, etc. The radio frequency transmit chain module is used to generate radio frequency pulses to excite a subject to generate a magnetic resonance signal, and a medical image can be reconstructed using the magnetic resonance signal.

A specific absorption ratio (SAR) of radio frequency energy is an important reference indicator in the transmit chain module, and shows the ratio of radio frequency signals absorbed by the scan subject. The SAR value is usually set below a certain threshold to avoid injury to the subject during a scanning process. In general, a patient having an implant, when undergoing magnetic resonance imaging, has a lower SAR limit. As medical technology advances, the number of patients with implants is increasing rapidly, and the manufacturers of these implants typically specify applicable medical scanning parameters for the implants.

When an MRI scanning system is used on different parts of a patient (typically different scan ranges determined in a scan procedure), an implant of the patient may also use a different scanning parameter, such that the scan procedure needs to be interrupted to reconfigure the scanning parameters separately, or even to create a new scan procedure, which may significantly disturb scanning and increase a time cost. Alternatively, when a patient has two or more implants, the applicable medical scanning parameters for these implants also need to be comprehensively configured.

### SUMMARY

One aspect of the present invention provides an MRI scanning method comprising: preconfiguring a plurality of scan configurations, and executing a scan procedure on a subject to be scanned using at least one of the preset plurality of scan configurations. The plurality of scan configurations correspond to a plurality of scan ranges of the subject to be scanned, and each of the scan configurations comprises an implant-related MRI scan parameter.

Another aspect of the present invention provides an MRI scanning system comprising: a controller, used to execute the MRI scanning method; and an MRI assembly, used to execute the scan procedure on the subject to be scanned on the basis of a control instruction of the controller.

Another aspect of the present invention provides an MRI scanning system comprising: an MRI assembly; A controller; and a graphical user interface interacting with the controller, the graphical user interface comprising a plurality of interfaces, the plurality of interfaces being used to display a plurality of preset scan configurations, respectively, the plurality of scan configurations corresponding to a plurality of scan ranges of a subject to be scanned, and each of the scan configurations comprising an implant-related MRI scan parameter, wherein the controller is used to control the MRI assembly to execute a scan procedure on the subject to be scanned using at least one of the preset plurality of scan configurations.

### BRIEF DESCRIPTION OF THE DRAWINGS

The described and other features, aspects, and advantages of the present invention will be better understood once the following detailed description has been read with reference to the accompanying drawings. In the accompanying drawings, the same reference signs are used to represent the same components throughout the accompanying drawings, in which:

FIG. 1 is an exemplary MRI scanning system according to some embodiments of the present invention;

FIG. 2 is an example of a subject to be scanned having an implant;

FIG. 3 is a conventional scan procedure for performing an MRI scan on a plurality of scan sites;

FIG. 4 is a flowchart of an MRI scanning method according to an embodiment of the present invention;

FIG. 5 is a block diagram of an MRI scanning system according to an embodiment of the present invention;

FIG. 6 is a block diagram of an MRI scanning system according to another embodiment of the present invention;

FIG. 7 is an example of an MRI scanning method according to embodiments of the present invention;

FIG. 8 is a flowchart of another embodiment of the MRI scanning method according to embodiments of the present invention;

FIG. 9 is a flowchart of an MRI scanning method according to another embodiment of the present invention;

FIG. 10 is an example of a graphical user interface in embodiments of the present invention;

FIG. 11 is a flowchart of an MRI scanning method according to another embodiment of the present invention;

FIG. 12 is a flowchart of an MRI scanning method according to another embodiment of the present invention;

FIG. 13 and FIG. 14 are body models acquired on the basis of different body metrics, respectively;

FIG. 15 is a flowchart of an MRI scanning method according to another embodiment of the present invention;

FIG. 16 is a body model at a second viewing angle;

FIG. 17 is a flowchart of an MRI scanning method according to another embodiment of the present invention;

FIG. 18 is a schematic view of adding an implant identifier to a body model using a medical image;

FIG. 19 is a flowchart of an MRI scanning method according to another embodiment of the present invention;

FIG. 20 is an example of safety prompt information displayed in a current interface;

FIG. 21 is another example of safety prompt information displayed in another interface; and

FIG. 22 is a flowchart of an MRI scanning method according to another embodiment of the present invention.

### DETAILED DESCRIPTION

Specific embodiments of the present invention will be described in detail below with reference to the accompanying drawings in order to assist those skilled in the art to understand exactly the subject matter set forth in the present invention. In the following detailed description of the following specific embodiments, the present specification does not describe in detail any known functions or configurations to prevent unnecessary details from affecting the disclosure of the present invention.

Unless otherwise defined, the technical or scientific terms used in the claims and the description should be as they are usually understood by those possessing ordinary skill in the technical field to which they belong. Terms such as “first”, “second”, and similar terms used in the present description and claims do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections. Furthermore, it should be understood that references to “an example” or “examples” of the present disclosure are not intended to be construed as excluding the existence of additional implementations that also incorporate the referenced features.

A “module”, “unit”, etc., as described herein may be implemented by using software, hardware, or a combination of software and hardware. For example, in accordance with some aspects of the embodiments of the present invention, the “modules” described herein may be implemented as computer program modules or circuit modules.

An “image” described herein may include a displayed image, or may include data that forms the displayed image.

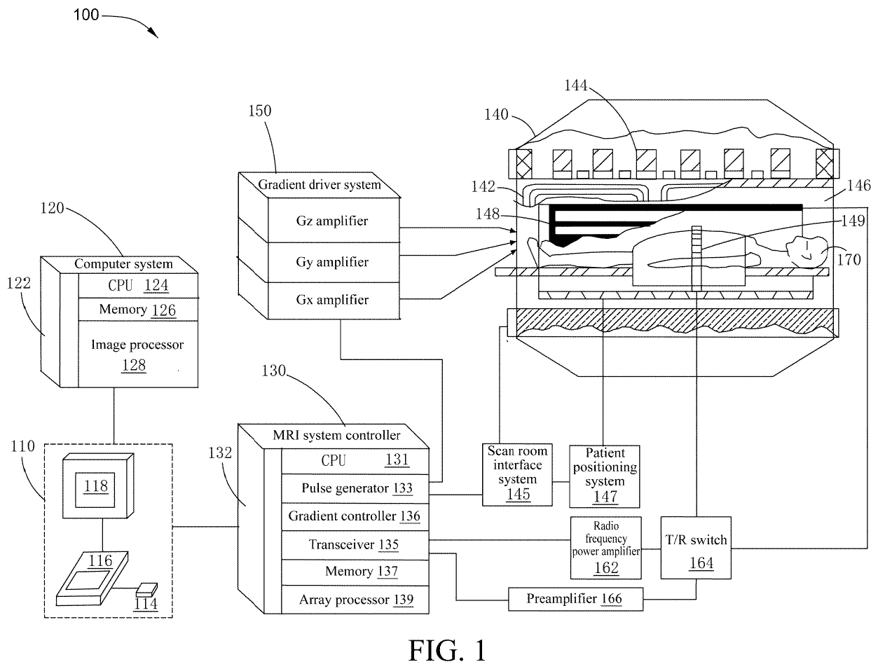

Referring to FIG. 1, an exemplary MRI scanning system 100 according to some embodiments of the present invention is illustrated. An operator workstation 110 is used to control the operation of the MRI system 100, the operator workstation 110 including an input apparatus 114, a control panel 116, and a display 118. The input apparatus 114 may be a joystick, a keyboard, a mouse, a trackball, a touch-activated screen, voice control, or any similar or equivalent input apparatus. The control panel 116 may include a keyboard, a touch-activated screen, voice control, a button, a slider, or any similar or equivalent control apparatus. The operator workstation 110 is coupled to and in communication with a computer system 120, and provides an interface to allow an operator to plan magnetic resonance scanning, display an image and a graphical user interface, perform image processing, and store data and images.

The computer system 120 includes a plurality of modules that communicate with one another by means of an electrical and/or data connection module 122. The connection module 122 may be a wired communication link, an optical fiber communication link, a wireless communication link, and the like. The computer system 120 may include a central processing unit (CPU) 124, a memory 126, and an image processor 128. In some embodiments, the image processor 128 may be replaced by an image processing function run in the CPU 124. The computer system 120 may be connected to an archive media apparatus, a persistent or backup memory, or a network. The computer system 120 may be coupled to and communicates with a separate MRI system controller 130.

The MRI system controller 130 includes a set of modules that communicate with one another by means of an electrical and/or data connection module 132. The connection module 132 may be a direct wired communication link, an optical fiber communication link, a wireless communication link, and the like. In an alternative embodiment, modules of the computer system 120 and the MRI system controller 130 may be implemented on the same computer system or on a plurality of computer systems. The MRI system controller 130 may include a CPU 131, a sequence pulse generator 133 that communicates with the operator workstation 110, a transceiver (or an RF transceiver) 135, a gradient controller 136, a memory 137, and an array processor 139.

A subject 170 of the MR scan may be positioned within a cylindrical imaging volume 146 of a magnetic resonance assembly 140 via a scanning table, and the subject 170 may have one or more implants in the body thereof. The MRI system controller 130 controls the scanning table to travel in a Z-axis direction of a magnetic resonance system, so as to deliver a predetermined site to be scanned of the subject 170 into the imaging volume 146. The magnetic resonance assembly 140 includes a superconducting magnet having a superconducting coil 144, a radio frequency (RF) coil assembly, and a gradient coil assembly 142. The superconducting coil 144 has a magnet aperture to form the cylindrical imaging volume 146. During operation, the superconducting coil 144 provides a static uniform longitudinal magnetic field B₀ throughout the cylindrical imaging volume 146. The radio-frequency coil assembly may include a body coil 148 and a surface coil 149, and may be used to send and/or receive a radio-frequency signal.

The MRI system controller 130 may receive a command from the operator workstation 110 to indicate a scan procedure to be executed during an MRI scan; in embodiments of the present invention, the scan procedure may include a plurality of scans for a plurality of scan ranges, respectively, each of the scans having a set scan configuration, and a plurality of scan configurations may be preconfigured on the basis of the embodiments of the present invention. Each of the scans is used to perform a predetermined scan sequence in the corresponding scan configuration; a “scan sequence” refers to a combination of pulses that have specific intensities, shapes, time sequences, etc. that are applied when a magnetic resonance imaging scan is executed. The pulses may typically include, for example, a radio-frequency pulse and a gradient pulse. A plurality of scan sequences may be pre-stored in the computer system 120, so that a sequence suitable for clinical examination requirements can be indicated by means of the operator workstation. The clinical examination requirements may include, for example, an imaging site, an imaging function, an imaging effect, scanning safety, and the like. The sequence pulse generator 133 of the MRI system controller 130 sends, on the basis of the indicated sequence, an instruction describing the time sequences, intensities, and shapes of the radio frequency pulse and gradient pulse in the sequence so as to operate a system component that executes the sequence.

A radio frequency pulse in the scan sequence sent by the pulse generator 133 may be generated by the transceiver 135, and the radio frequency pulse is amplified by a radio frequency power amplifier 162. The amplified radio frequency pulse is provided to the body coil 148 via a transmit/receive switch (T/R switch) 164, and the RF body coil 148 then immediately provides a transverse magnetic field B₁. As a non-limiting example, a transmitting portion of the transceiver 135, the radio frequency power amplifier 162, the T/R switch 164, and the like constitute at least a portion of a radio frequency transmit link. The transverse magnetic field B₁ is substantially perpendicular to B₀ throughout the cylindrical imaging volume 146, and the transverse magnetic field B₁ is used to excite stimulated nuclei within the body of the scan subject, thereby generating an MR signal.

A portion of the energy loaded by the radio frequency pulse is released in the form of heat, which is absorbed by the human body, and over time, the energy is deposited at a scan site of the human body, thereby resulting in a local or systemic temperature increase of the human body. The energy that can be absorbed per unit of time and per unit of body weight is defined as SAR, and different SAR values are usually set for different parts of the body.

The gradient pulse in the scan sequence sent by the pulse generator 133 may be generated by the gradient controller 136 and acts on a gradient driver 150. The gradient driver 150 includes Gₓ, G_{y}, and G_{z} amplifiers, and the like. Each of the G_{y}, G_{y}, and G_{z} gradient amplifiers is used to excite a corresponding gradient coil in the gradient coil assembly 142, so as to generate a magnetic field gradient used to spatially encode an MR signal during an MR scan.

The pulse generator 133 is coupled to and communicates with a scan room interface system 145, and the scan room interface system 145 can receive signals from various sensors associated with the state of the magnetic resonance assembly 140 and various processors provided in a scan room. The scan room interface system 145 is further coupled to and communicates with a patient positioning system 147, the patient positioning system 147 sending and receiving a signal to control the scanning table to travel so as to transport the patient or the subject 170 to a desired position to perform the MR scan.

As described above, the RF body coil 148 and the RF surface coil 149 may be used to transmit a radio frequency pulse and/or receive MR signals from the scan subject. The MR signals emitted by excited nuclei in the body of the scan subject may be sensed and received by the RF body coil 148 or the RF surface coil 149 and then sent back to a preamplifier 166 by means of the T/R switch 164. The T/R switch 164 may be controlled by a signal from the MRI system controller 130 to electrically connect, during a transmit mode, the radio-frequency power amplifier 162 to the RF body coil 148 and to connect, during a receive mode, the preamplifier 166 to the RF body coil 148. The T/R switch 164 may further enable the RF surface coil 149 to be used in the transmit mode or the receive mode.

In some embodiments, the MR signals sensed and received by the RF body coil 148 or the RF surface coil 149 and amplified by the preamplifier 166 are demodulated, filtered, and digitized in a receiving portion of the transceiver 135, and transmitted as a raw k-space data array to the memory 137 in the MRI system controller 130.

A reconstructed magnetic resonance image may be obtained by transforming/processing the stored raw k-space data. For each image to be reconstructed, the data is rearranged into separate k-space data arrays, and each of the separate k-space data arrays is input into the array processor 139, the array processor being operated to transform the data into an array of image data.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and MRI system controller 130 may be implemented on the same computer system or on a plurality of computer systems. It should be understood that the MRI system 100 shown in FIG. 1 is intended for illustration. Suitable MRI systems may include more, fewer, and/or different components.

The MRI system controller 130 and the image processor 128 may separately or jointly include a computer processor and a storage medium, the storage medium recording a program for predetermined data processing that is to be executed by the computer processor. For example, the storage medium may store a program used to implement scanning processing (such as a scan parameter configuration, a scan control procedure, an imaging sequence), image reconstruction, image processing, and the like. Specifically, the storage medium may store the MRI scanning method according to any embodiment of the present invention. The described storage medium may include, for example, a ROM, a floppy disk, a hard disk, an optical disk, a magneto-optical disk, a CD-ROM, or a non-volatile memory card.

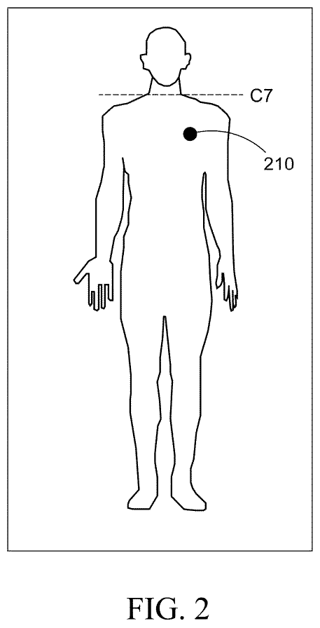

When an MRI scan is performed on a subject to be detected, a complete scan may include scanning a plurality of sites of the subject, and thus the complete scan may involve scanning at different scan ranges. In some scenarios, for example, when the subject has a certain implant, the implant has different scan configuration requirements at different scan ranges. As shown in FIG. 2, there is a cardiac pacemaker 210 which requires that a RF field root mean square value B+rms must be less than or equal to 3.2 uT (micro Tesla) when a scan range of an MRI scan (or a site to be scanned) is above a cervical C7 vertebra, and the RF field root mean square B+rms must be less than or equal to 2.8 uT when the scan range is below the vertebra.

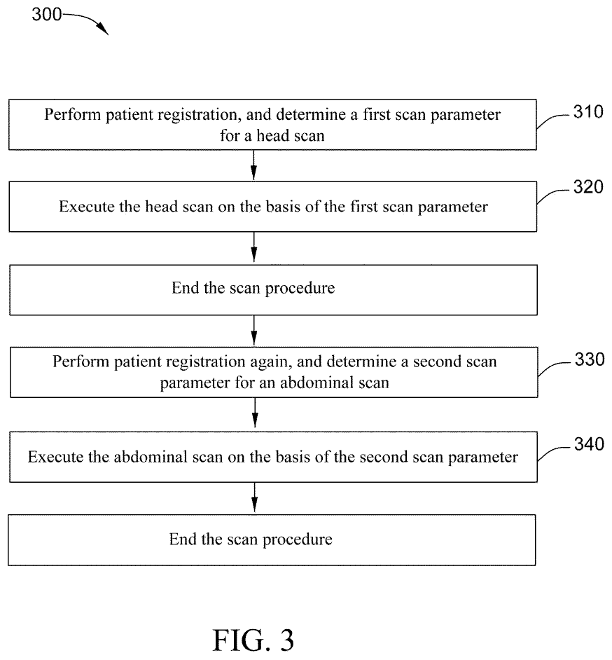

During a complete scan, when the whole body of a subject needs to be scanned, or a site above the C7 vertebra (e.g., the head) and a site below the C7 cone (e.g., the abdomen) need to be scanned separately, a conventional scan procedure 300 is shown in FIG. 3. In step 310, patient registration is performed, and a first scan parameter is determined for a head scan. In step 320, the head scan is performed on the basis of the first scan parameter, and the scan procedure ends, the head scan including moving a scanning table such that the head region of the subject is in alignment with a scan center for imaging. In step 330, patient registration is performed again, and a second scan parameter is determined for an abdominal scan. In step 340, the abdominal scan is performed on the basis of the second scan parameter, and the scan procedure ends, the abdominal scan including moving the scanning table such that the abdominal region of the subject is in alignment with the scan center for imaging.

This approach requires repeated patient registration (which may include, for example, inputting/editing patient information and identifying a patient from a registration list) and repeated configuration of scan parameters, interrupting the scanning process, and more time cost is wasted when the number of first or second scans is greater.

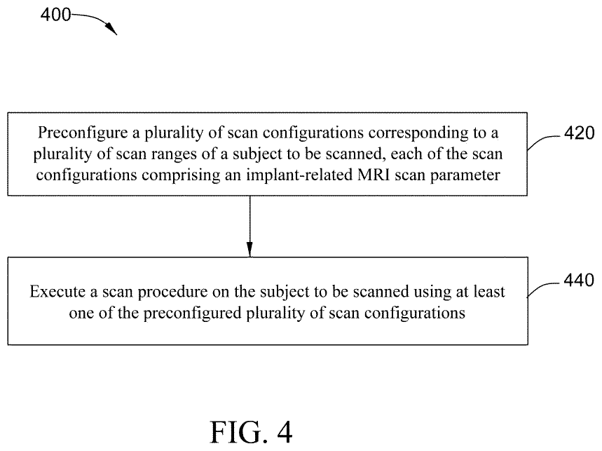

FIG. 4 shows a flowchart 400 of an MRI scanning method according to an embodiment of the present invention. In step 420, a plurality of scan configurations are preconfigured, the plurality of scan configurations corresponding to a plurality of scan ranges of a subject to be scanned, and each of the scan configurations including an implant-related MRI scan parameter. In step 440, a scan procedure is executed on the subject to be scanned using at least one of the preconfigured plurality of scan configurations.

The “scan procedure” described above may include one or more individual scanning processes for one or more scan ranges or sites of the subject to be tested; for example, it may include some or all of the scans executed in a period of time during the subject to be tested is positioned in and out of the scanning table.

The “preconfiguration” described above indicates that scan configuration information is configured before the scan procedure is performed; for a plurality of individual scanning processes in the scan procedure (e.g., having different scan ranges), the scan configurations used in all the individual scanning processes may be determined before the first individual scanning process is executed, e.g., implant-related MRI scan parameters are configured in each individual scanning process. Therefore, when the scan procedure is executed, any one of the individual scanning processes may be executed directly on the basis of the preconfigured corresponding scan configuration; for example, a plurality of individual scanning processes may be executed sequentially, without performing scan configuration or configuring scanning parameters between two adjacent individual scanning processes.

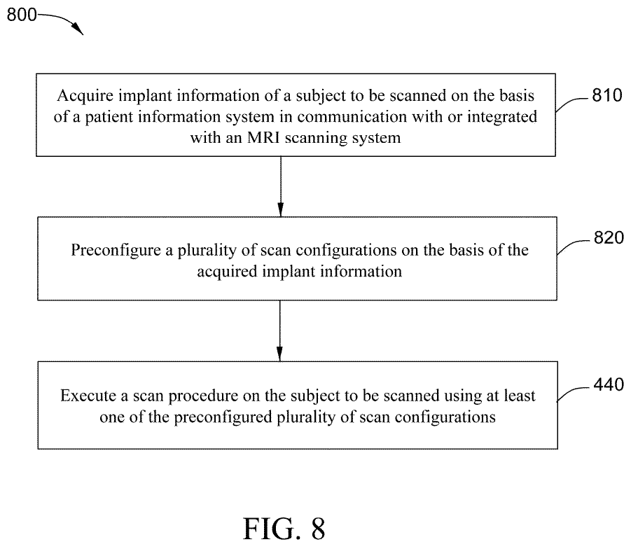

The “implant-related scanning parameter” described above refers to a parameter and/or hardware configuration whose parameter value may be directly or indirectly limited by the implant; for example, this may include at least one of SAR (Specific Absorption Rate, which may include a whole body SAR value (WB SAR) and a local SAR value (e.g., Head SAR)), an allowed RF transmit coil, an allowed RF drive mode, a max single series time, a spatial gradient, a max slew rate, and the aforementioned RF field root mean square value (B+rms), and the like. A set of these parameters/hardware configurations is also shown in the graphical user interface shown in FIG. 8 as an example.

In an embodiment of the present invention, the plurality of scan configurations may include a first scan configuration and a second scan configuration, and the scan procedure may include: sequentially performing a first scan and a second scan on the basis of the first scan configuration and the second scan configuration, respectively.

“Sequentially performing a first scan and a second scan” as described above means that, after the first scan is completed, the second scan can be started by means of a user operation or an automatic control without requiring configuring the scan configuration again.

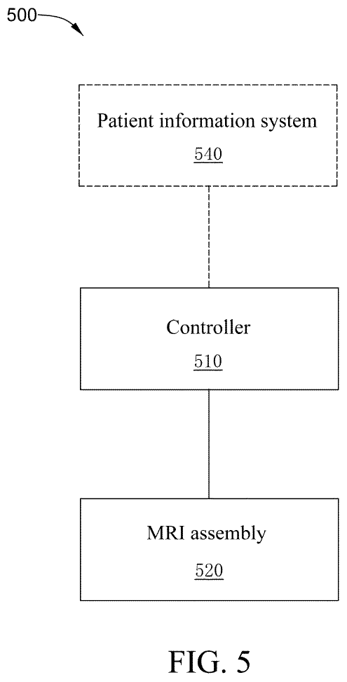

FIG. 5 is a block diagram of an MRI scanning system 500 according to an embodiment of the present invention, wherein the MRI scanning system includes a controller 510 and an MRI assembly 520. The controller 510 is used to execute the MRI scanning method according to any one of the embodiments of the present invention, and the MRI assembly 520 is used to execute the scan procedure on the subject to be scanned on the basis of a control instruction of the controller 510.

For example, the controller 510 may be used to execute: preconfiguring the plurality of scan configurations, and performing a scan procedure on a subject to be scanned using at least one of the preconfigured plurality of scan configurations.

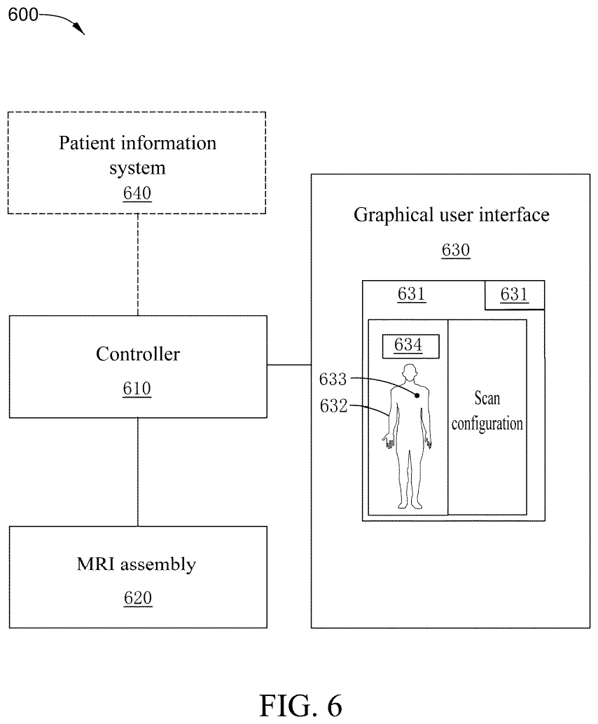

FIG. 6 is a block diagram of an MRI scanning system 600 according to another embodiment of the present invention, wherein the MRI scanning system includes an MRI assembly 620, a controller 610 and a graphical user interface 630 interacting with the controller 610. The graphical user interface 630 includes a plurality of interfaces 631, the plurality of interfaces being used to display the preset plurality of scan configurations, respectively. The controller 610 is used to control the MRI assembly 620 to execute a scan procedure on the subject to be scanned using at least one of the preconfigured plurality of scan configurations.

Further, each of the plurality of interfaces 631 is also used to: display a body model 632 matching the subject to be scanned; receive a user operation to add an implant identifier 633 to the body model; and display safety prompt information 634 in a corresponding scan configuration, the safety prompt information being generated on the basis of position information of the implant identifier 633 in the body model 632.

In the embodiments of the present invention, the controller 510, 610 may be at least partially in communication with the computer system 120 and the MR system controller 130 in FIG. 1, or include at least a part of the computer system 120 and the MR system controller 130, or be integrated with at least a part of the computer system 120 and the MR system controller 130, respectively. The MRI assembly 520, 620 may also include part or all of the MRI assembly 140 in FIG. 1. The graphical user interface 630 may be displayed via a display 118 or a display screen provided on the MRI assembly 520, 620.

As shown in FIG. 5 and FIG. 6, optionally, the controller 510, 610 may also be in communication with a patient information system 540 via a remote or local network, and in some embodiments, the patient information system 540 may be integrated with an MRI scanning system. The patient information system 540 will be further introduced below in the further description of the MRI scanning method according to the embodiment of the present invention.

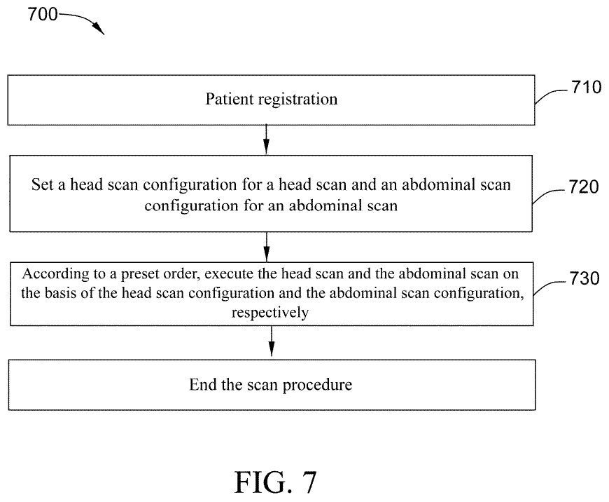

FIG. 7 is an example 700 of an MRI scanning method according to embodiments of the present invention. In step 710, patient registration is performed, and in this registration, an information input may be performed via the MRI scanning system 500, 600 of the present invention, and registered patient information may subsequently be stored in the MRI scanning system 500 and the patient information system 540. In step 720, a head scan configuration is configured for a head scan and an abdominal scan configuration is configured for an abdominal scan, and one or more parameters in the head scan configuration and the abdomen scan configuration may have different set values. Step 720 is performed before the scan procedure is executed, i.e., before step 730. In step 730, the head scan and the abdominal scan are executed on the basis of the head scan configuration and the abdominal scan configuration, respectively, in a preset order, wherein a plurality of head scans and/or a plurality of abdomen scans may be performed separately or alternately, and every two adjacent scans may be used as the first scan and the second scan.

In this way, it can be avoided that after one scan (e.g., a head scan) is completed, the scan procedure is interrupted to configure a scan configuration for the next scan (e.g., abdomen), thereby improving scanning efficiency.

FIG. 8 is a flowchart of another embodiment of the MRI scanning method according to embodiments of the present invention, wherein step 420, i.e., presetting a plurality of scan configurations, may be implemented by means of steps 810 and 820. In step 810, implant information of a subject to be scanned is acquired on the basis of the patient information system 540 in communication with or integrated with an MRI scanning system. At step 820, the plurality of scan configurations are preconfigured on the basis of the acquired implant information.

The aforementioned patient information system may be used to register, store, and access patient information, and may be one or more of a Hospital Information System (HIS), a Radiology Information System (RIS), an Electronic Medical Record (EMR) (or EHR), an EPIC medical management system, etc.

“In communication with” may include a local network link or a remote network connection.

The implant information of the subject to be scanned acquired at step 810 may include, for example, one or more of implant type, model, size, scan configuration information applicable for different scan ranges, etc. The implant information may be stored in the MRI scanning system, and in some embodiments, a plurality of scan configurations for different scan ranges may be automatically generated on the basis of the implant information, and confirmed or adjusted by a user. In some embodiments, the scan configurations may also be performed directly via a user operation, e.g., the user creates a plurality of scan configurations in a graphical user interface by means of the acquired implant information.

In the embodiments of the present invention, preconfiguring a plurality of scan configurations may include at least one of the following operations: deleting at least one existing scan configuration; editing at least one existing scan configuration; and adding a new scan configuration.

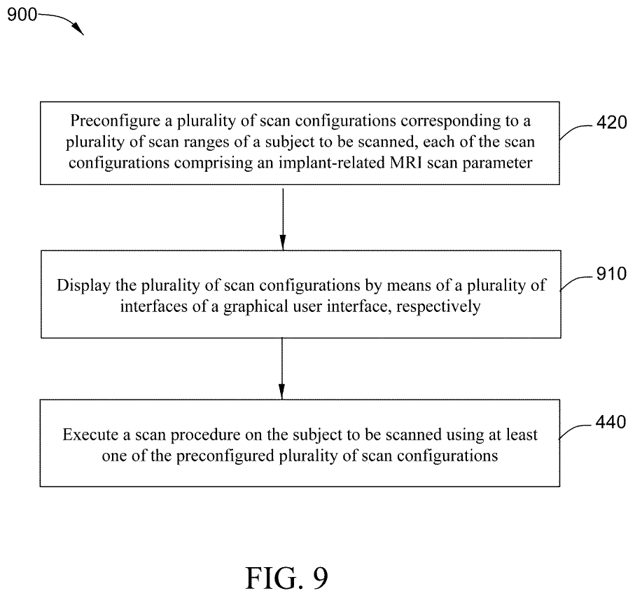

FIG. 9 is a flowchart of an MRI scanning method according to another embodiment of the present invention, further including step 910: displaying the plurality of scan configurations by means of a plurality of interfaces of a graphical user interface, respectively; the graphical user interface may include the graphical user interface 630 shown in FIG. 6.

Further, in step 420, the plurality of scan configurations may be configured on the basis of a user input received via the graphical user interface.

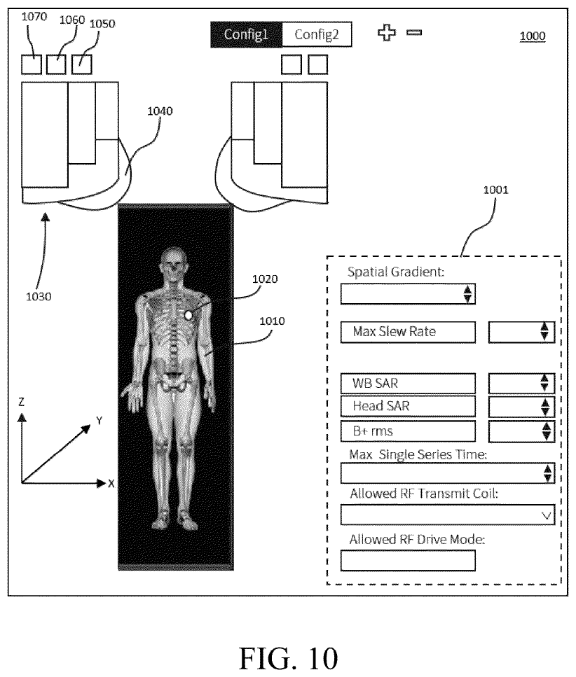

FIG. 10 is an example 1000 of the graphical user interface 630, including a first interface Config1 and a second interface Config2, wherein detailed information of the first interface Config1 is displayed in the current interface, for example, including a scan configuration 1001 and a body model 1010, and may further include an implant identifier 1020 added to the body model 1010. The detailed information may further include a simulation model 1030 of the MRI scanning system, an implant safety constraint region 1040, and the body model 1010 in the current state, as well as the relative positions therein of the implant identifier 1020 and the implant safety constraint region 1040. The current interface may be switched to display detailed information of the second interface Config2 by means of clicking an icon of the second interface Config2. In other embodiments, the first interface and the second interface may be displayed simultaneously in different regions of the current interface.

The first interface or the second interface may be created on the basis of a user operation (e.g., clicking on an “add” icon “+” in FIG. 10), and the user may also delete the current interface (e.g., the first interface) by operating a “delete” icon “−”, or modify/edit any item of information in the corresponding scan configuration in the current interface.

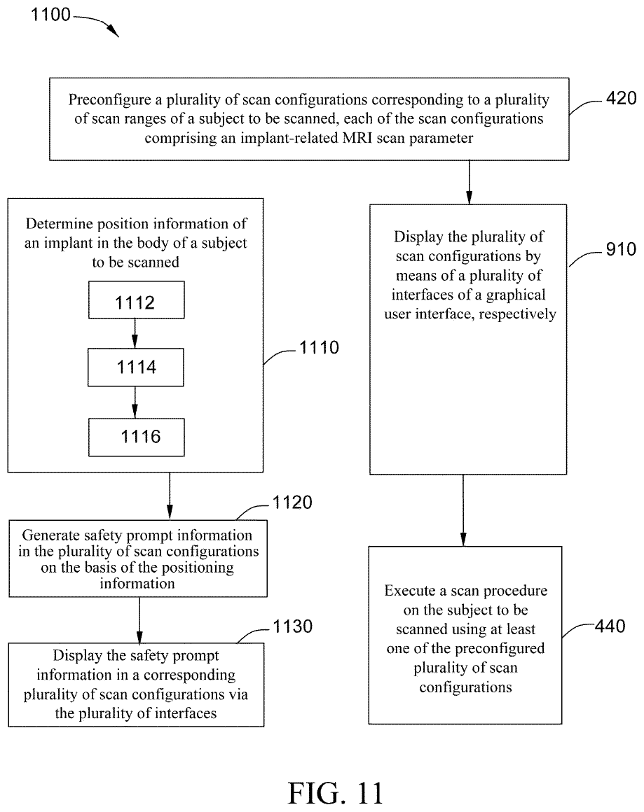

FIG. 11 is a flowchart 1100 of an MRI scanning method according to another embodiment of the present invention, the method further including steps 1110, 1120 and 1130. In step 1110, position information of an implant of a subject to be scanned is determined in a body. In step 1120, safety prompt information in the plurality of scan configurations is generated on the basis of the position information. In step 1130, the safety prompt information in a corresponding plurality of scan configurations is displayed via the plurality of interfaces.

Step 1110 may further include steps 1112, 1114 and 1116. In step 1112, the body model 1010 matching the subject to be scanned is displayed via the graphical user interface (as shown in FIG. 10). In step 1114, the implant identifier 1020 is added to the body model 1010. In step 1116, position information of the implant identifier 1020 relative to the body model 1010 is determined.

The position information of the implant identifier 1020 relative to the body model 1010 can reflect the actual position of the implant in the body, and therefore, in step 1120, the safety prompt information may be generated on the basis of the position information of the implant identifier 1020 relative to the body model 1010. Based on the actual position of the implant of the subject to be tested in the body, it is determined whether a safe scan can be executed in the current scan configuration, and a safety evaluation result is presented to the user by means of displaying the safety prompt information, so that a scanning risk is predicted before actual scanning, thereby preventing the occurrence of a safety incident.

The body model 1010 may be a three-dimensional image model having a human-like structure; for example, the body model 1010 may include, but is not limited to, a torso, limbs, head, brain, heart, liver, spleen, stomach, bone, etc., to facilitate the addition of implant identifiers at more accurate positions. In some embodiments, the three-dimensional body model 1010 may have a more simplified structure, e.g., may include only an outer contour, and a user may perform an operation in the simplified three-dimensional body model on the basis of their own knowledge of human anatomy. The body model may be a uniform standard model or selected from a plurality of standard models. In order to improve the accuracy of a security evaluation, the body model 1010 may be obtained after being matched with the subject to be scanned, and said “matching” may include matching or being similar to the subject to be scanned in terms of height, weight, body proportions, gender, age, etc. In some embodiments, “matching” includes performing zooming, proportional adjustment and the like on all or part of the body model. In an embodiment of the present invention, the body model may be adjusted by means of an adjustment icon 1070 in an operation interface.

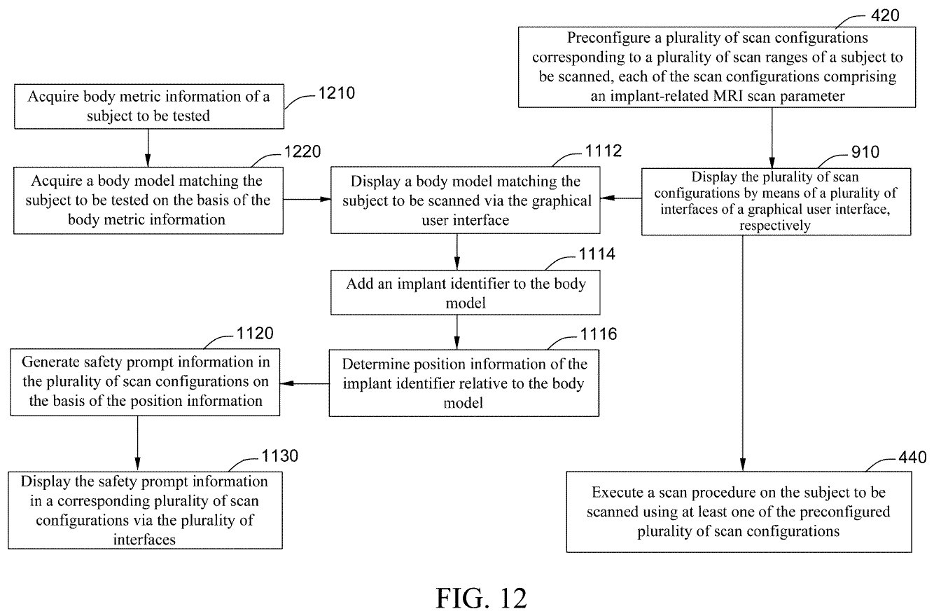

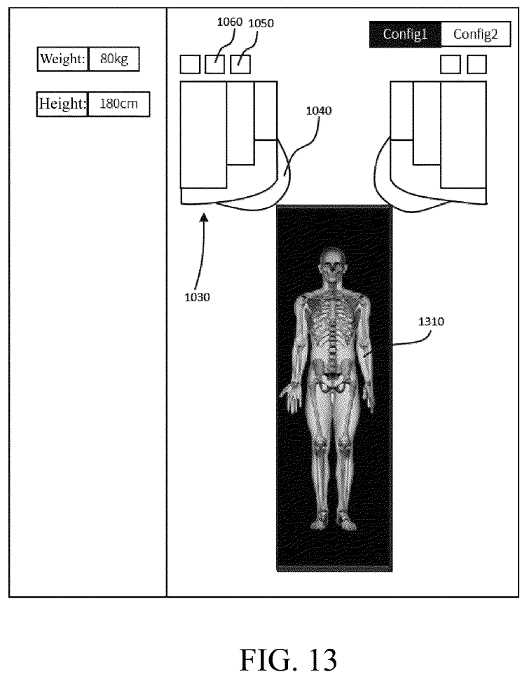

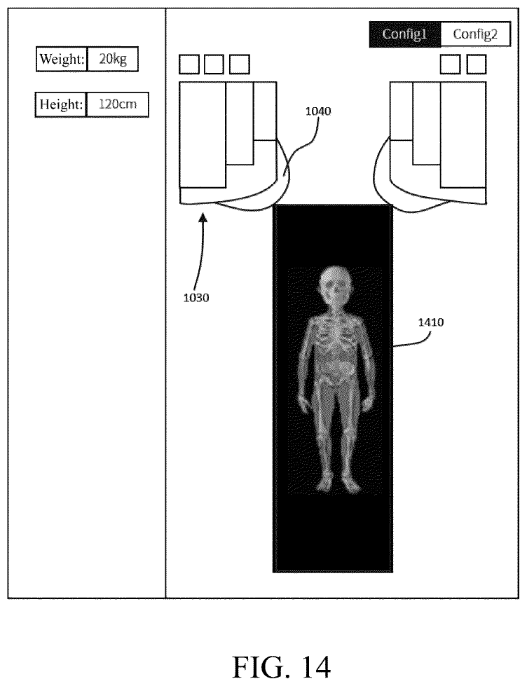

FIG. 12 is a flowchart of an MRI scanning method according to another embodiment of the present invention, and FIG. 13 and FIG. 14 are body models acquired on the basis of different body metrics, respectively. An example of acquiring a body model matching a subject to be scanned will be described below, with reference to FIGS. 12-14.

In step 1210, body metric information of the subject to be tested is acquired, wherein the body metric information may include at least one of height and weight, and the body metric information may be acquired by asking the subject to be scanned or by measuring by any technical means. In step 1220, a body model matching the subject to be tested is obtained on the basis of the body metric information. In one example, the body model may be obtained by automatically adjusting a prestored standard body model on the basis of the body metric information, the standard body model possibly including a basic human structure. In another example, a body model matching the body metric information may also be selected from a plurality of prestored standard body models. For example, a plurality of standard models based on different height ranges and weight ranges may be stored so that an appropriate body model can be quickly selected. In other examples, the prestored plurality of standard body models may also be obtained by combining significant features such as age, gender, body type, etc., so that the accuracy of matching is improved. In other examples, approximate matching may first be performed by selecting a standard model, and exact matching may be performed by adjusting the selected standard model, wherein the adjustment may include, for example, further zooming, proportion adjustment, etc.

The body model 1310 in FIG. 13 is obtained based on body metrics such as body weight (80 kg) and body height (180 cm), and the body model 1410 in FIG. 14 is obtained based on body metrics such as body weight (20 kg) and body height (120 cm). Therefore, it is possible to obtain a body model that better matches the actual features of the subject to be tested, so that the position information of the implant identifier added to the body model is more accurate, thereby facilitating a safety prediction for the implant. As shown in FIGS. 13 and 14, the body metric information may be displayed and input by means of a corresponding interface of each scan configuration.

In other embodiments, a three-dimensional image of a subject to be tested may also be acquired by photographing the current subject to be scanned via a three-dimensional camera, and a body model matching the subject to be scanned may be generated on the basis of the three-dimensional image. For example, a subject to be scanned on a scanning table may be photographed, the photographed image may be preprocessed, and the preprocessed image may be used as the body model; or, the subject to be scanned may be photographed in any position, and the photographed image may be subjected to posture correction, coordinate transformation, or the like to generate the body model. Since the body model is generated on the basis of an image of the subject itself, subsequently obtained implant position information will be more accurate.

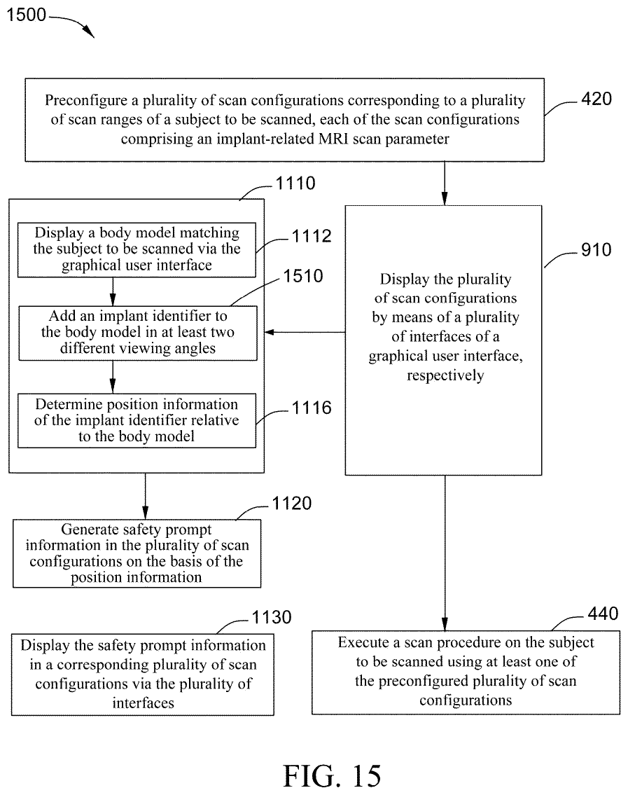

The implant identifier 1020 may be added to the body model 1010 (which may include, for example, the body model 1310 in FIG. 13 or the body model 1410 in FIG. 14) in the current interface by various means; for example, according to the implant information acquired from the patient information system 540 described above, if it is determined that the subject to be scanned has a cardiac pacemaker, the implant identifier may be added at the cardiac site of the body model 1010 based on an operation indication of the graphical user interface. An embodiment of adding an implant identifier will be described in detail below with reference to FIGS. 15-17.

FIG. 15 is a flowchart of an MRI scanning method according to another embodiment of the present invention, wherein step 1114 may further include step 1510: adding an implant identifier to the body model in at least two different viewing angles, respectively.

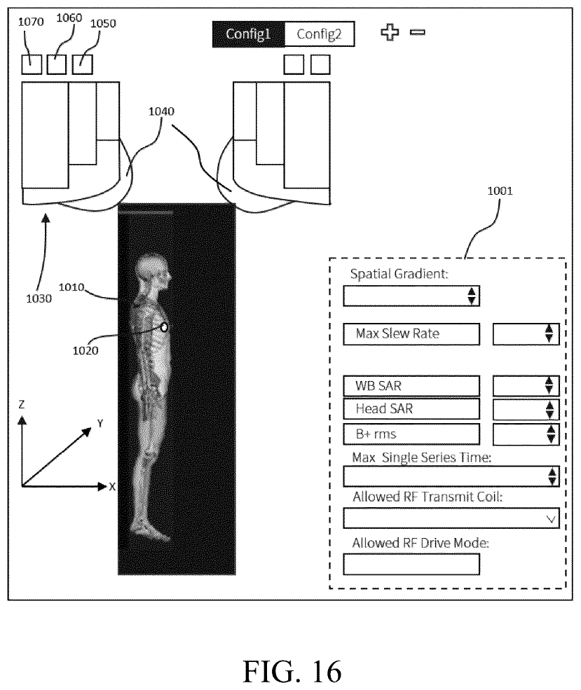

FIG. 10 is the body model 1010 at a first viewing angle (front viewing angle), and FIG. 16 is the body model 1010 at a second viewing angle (side viewing angle), wherein the first viewing angle and the second viewing angle can be switched by means of a viewing angle switching icon 1050 in the operation interface. In step 1510, the implant identifier 1020 may be first added to the body model 1010 in the default first viewing angle, so that the position information of the implant identifier 1020 in an X-Z plane can be determined, and then, on the basis of an indication of an operation of the viewing angle switching icon 1050, the body model 1010 in the second viewing angle is displayed, as shown in FIG. 16, the body model 1010 in the second viewing angle already having the implant identifier 1020 positioned in the X-Y plane, and the position of the implant identifier 1020 in a Y direction may be further adjusted so that the three-dimensional position information of the implant identifier can be determined.

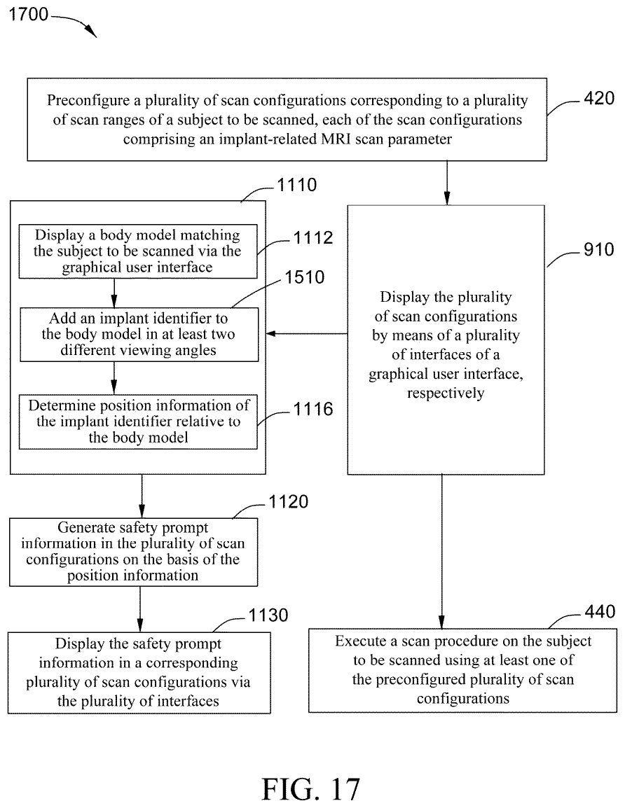

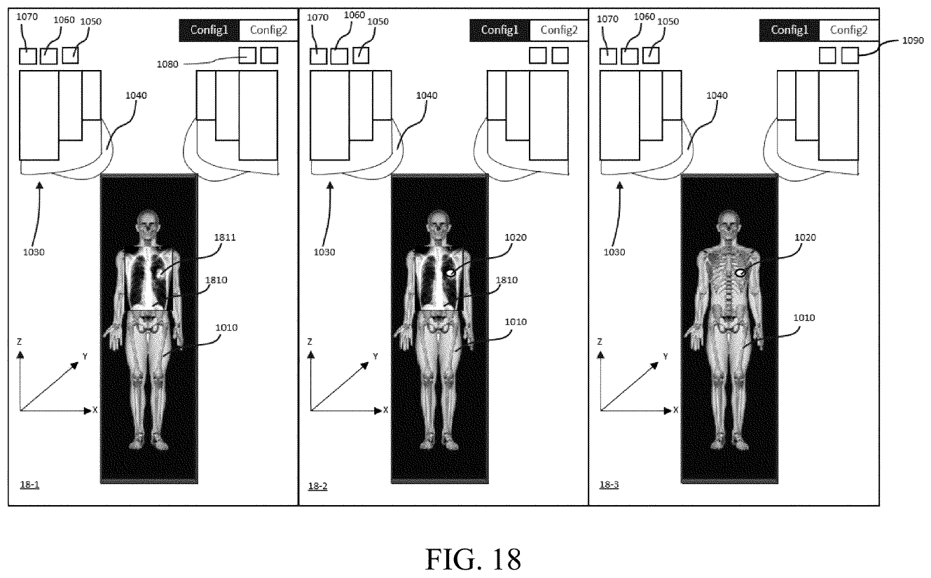

FIG. 17 is a flowchart 1700 of an MRI scanning method according to another embodiment of the present invention, wherein step 1114 may further include steps 1720, 1730, 1740 and 1750. FIG. 18 is a body model 1010 of a subject to be scanned and a medical image 1810 of the subject to be scanned. Another example of adding an implant identifier will be described below with reference to FIGS. 17 and 18.

In step 1720, the medical image 1810 of the subject to be scanned is acquired on the basis the patient information system 540 described above, the medical image 1810 may be an image of an imaging modality such as MRI, CT, ultrasound, PET, or any combination thereof, the medical image 1810 is acquired by imaging an image to be scanned in a previous diagnosis/examination, and the medical image 1810 contains an implant 1811 that can be identified by a device or the naked eye.

In step 1730, the medical image 1810 and the body model 1010 displayed at step 1112 are matched. The “matching” 8 may allow the size of the body part in the medical image 1810 to be matched with the size of the body model 1010, e.g., may cover or coincide with a corresponding body part of the body model 1010, which may be achieved by adjusting either the medical image 1810 or the body model 1010.

In step 1740, the position information of the implant is determined in the medical image 1810. For example, the implant in the medical image 1810 may be indicated by means of adding an auxiliary identifier at the position of the implant. The auxiliary identifier may be a wire frame, a coil, a dot, or other shaped identifier. The position information of the implant may also be determined in the medical image by means of image detection techniques, or the implant identifier 1020 may be directly placed at the position of the implant in the medical image 1810.

In step 1750, the implant identifier 1020 is added at a corresponding position of the body model on the basis of the determined position information of the implant; for example, the implant identifier 1020 may be added at a position corresponding to the implant position information on the body model by means of an operation indication of a user on a graphical user interface, or the implant identifier may be directly generated at a mapping position of the body model 1010 after the position information of the implant is determined, or the auxiliary identifier/implant identifier may be directly mapped in the body model 1010.

As shown in FIG. 18, after the medical image 1810 of the chest of the subject to be scanned is matched with the chest of the body model 1010, a display interface is as shown in 18-1 of FIG. 18, the implant 1811 being displayed on the right side of the image (corresponding to the position of the heart in the body), and the user can add the implant identifier 1020 by, for example, clicking a right mouse button at the position of the displayed implant 1811, so as to form the image shown in 18-2 of FIG. 18. The medical image is then removed from the interface or only removed from the body model 1010, and the added implant identifier 1020 in the interface and the position of the body model 1010 remain unchanged, to form the image shown in 18-3 of FIG. 18, i.e., the addition of the implant identifier to the body model is achieved.

While 18-1 of FIG. 18 only shows the medical image matched with the body model at the front viewing angle, in order to add the implant identifier in a more precise position, the position information of the implant may also be determined at other viewing angles, which may be implemented in connection with the solutions described in FIGS. 15-16 and 17-18. For example, in step 1730, matching the medical image with the body model may specifically include: acquiring medical images in at least two different anatomical orientations, respectively, and matching the medical images in the at least two different anatomical orientations with the body model in at least two different viewing angles, respectively.

In an example of the present invention, the medical image 1810 may be obtained by a “reference image importing” icon 1080 in the operation interface, and the medical image 1810 may be hidden or removed by a “reference image hiding” icon 1090 in the operation interface.

Adding an implant identifier in the body model in any of the above embodiments enables determination of position information of the implant identifier relative to the body model, wherein the position information can be used for determining position information of the implant of the subject to be scanned in the body. Therefore, in step 1120, safety prompt information in a plurality of scan configurations may be generated on the basis of the positioning information.

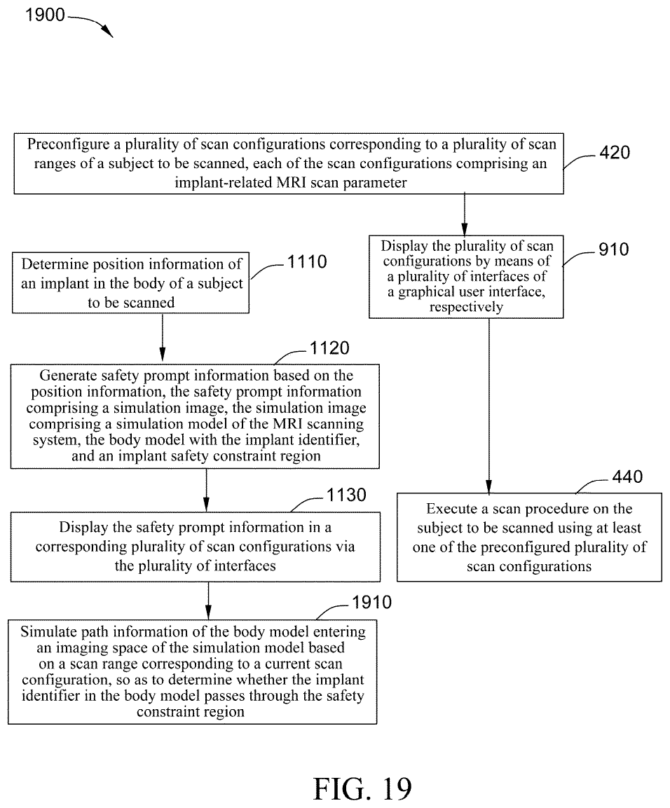

Referring to FIG. 19, a flowchart 1900 of an MRI scanning method according to another embodiment of the present invention is shown, wherein, in step 1120, the safety prompt information generated on the basis of the determined position information of the implant of the subject to be scanned in the body includes a simulation image. The simulation image may include a simulation model of an MRI scanning system, the body model having the implant identifier, and an implant safety constraint region.

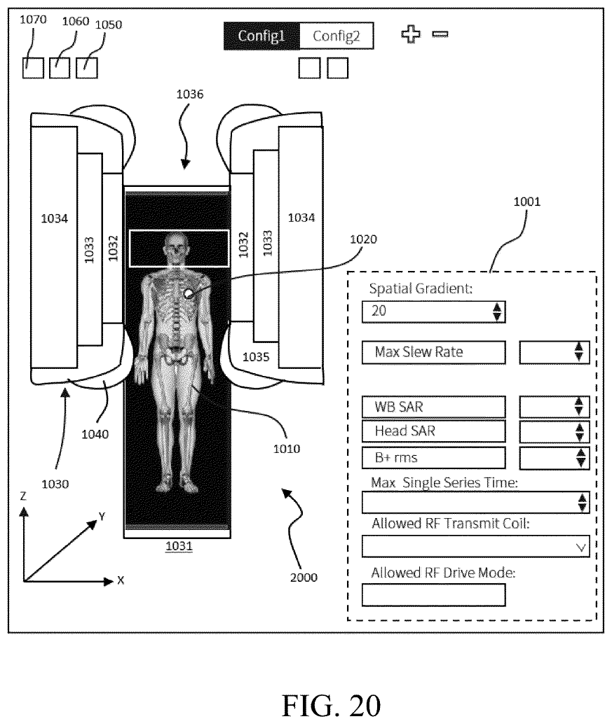

Referring to FIG. 20, an example 2000 of the safety prompt information 634 is shown displayed in a current interface (e.g., first interface Config1). In particular, the body model 1010, the implant identifier 1020 located in the body model, the simulation model 1030 of the MRI scanning system, and the implant safety constraint region (e.g., arc-shaped regions at both ends of a magnet housing) 1040 are shown.

The simulation model 1030 may be pre-generated according to at least some components of the MRI scanning system, e.g., comprising a simulated scanning table 1031, body coil 1032, gradient coil 1033, main magnet 1034, magnet housing 1035 and the like, a simulated imaging space 1036 being formed in a central region of the simulated body coil 1032, and the simulated imaging space 1036, e.g., corresponding to the imaging volume 146 shown in FIG. 1.

The implant safety constraint region 1040 may be determined on the basis of the capability of the current implant to withstand a spatial gradient; for example, in FIG. 20, when a limit of the spatial gradient is 20 T/m, the implant safety constraint region 1040 is a region having a spatial gradient greater than 20 T/m, and the safety prompt information determines and shows relative position information between the implant identifier 1020 and the safety constraint region 1040 in the Z direction (the length direction of the scanning table), so that it can be predicted whether the implant can avoid the safety constraint region 1040 while traveling in the Z direction along with the scanning table.

Therefore, as shown in FIG. 19, the MRI scanning method may further include: step 1910, simulating path information of the body model 1010 entering the imaging space 1036 of the simulation model 1030 of the MRI scanning system on the basis of the scan range corresponding to the current scan configuration, so as to determine whether the implant identifier 1020 in the body model 1010 passes through the safety constraint region 1040.

For example, if the scan range in the current configuration is a head region, a traveling path of the head region 1011 of the body model 1010 from an initial position to the imaging space of the simulation model 1030 is predicted according to a process of positioning the subject to be scanned at the time of actual scanning (i.e., the scanning table is moved from the initial position to a scan position in which the head region of the subject to be tested is located in the imaging space, or the center of the head region is aligned with a scan center), and it is predicted whether the implant identifier 1020 will enter the implant safety constraint region 1040 when traveling along the path.

In some embodiments, the results of the “prediction” or “simulation” described above may be presented to the user via a graphical user interface in the form of a still image that may display a simulated state of the scan range (e.g., the head region 1011) after being positioned in the imaging space 1036, e.g., an image on the left side of FIG. 20 is used to present the simulation results described above, wherein the center of the head region is aligned with the scan center, and an operator may also determine whether it is safe based on viewing a distance between the implant identifier 1020 and the safety constraint region 1040 in the X direction. In other embodiments, the body model 1010 in the still image may also be at other positions, such as the initial position.

In some embodiments, the results of the “prediction” or “simulation” described above may also be presented to the user via a graphical user interface in the form of a video, in which a process of positioning the body model in the imaging space 1036 relative to the scanning range (e.g., head region 1011) into which the simulation model 1030 travels from the initial position is displayed, and a distance between the implant identifier 1020 and the implant safety constraint region 1040 during said travel is displayed.

In some embodiments, in the described still image or video, a specific value of the distance between the implant identifier 1020 and the implant safety constraint area may be further shown, so that the user can more intuitively determine whether it is safe.

In one example, the path information of the scanning table 1031 traveling from an initial position (e.g., the position of the body model 1010 relative to the imaging space 1036 in FIG. 10) to a target position (e.g., the position of the body model 1010 relative to the imaging space 1036 in FIG. 20) may be simulated by means of a “simulation” icon 1060 in the operator interface, so as to generate safety prompt information 2000.

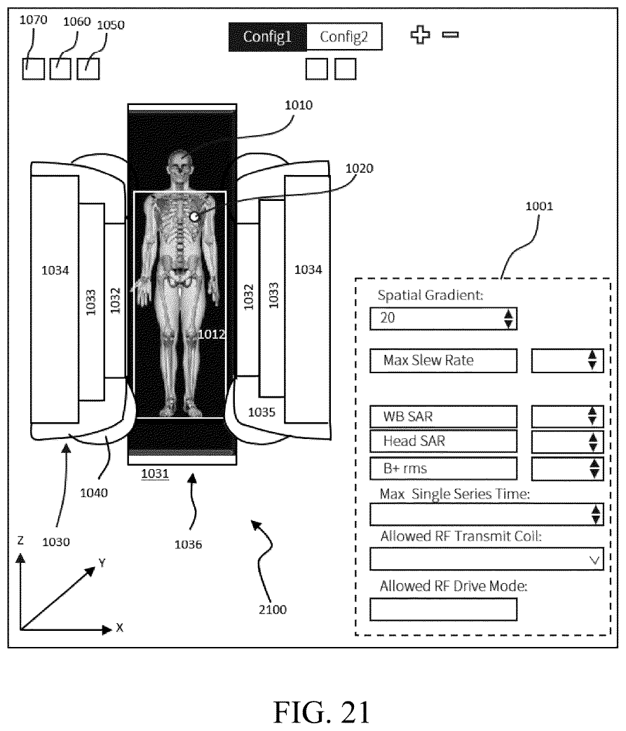

Although the safety prompt information 2000 in one scan configuration is shown in detail by only one interface in FIG. 20, it may be switched to safety security prompt information in another scan configuration by operating the graphical user; for example, FIG. 21 shows an interface when the user clicks an icon “Config2,” in which detailed information of another scan configuration and security prompt information 2100 in the other scan configuration are shown, wherein, in FIG. 21, a scan range 1012 (e.g., lower body) of the body model 1010 is positioned in the imaging space 1036 of the simulation model 1030, and thus, at step 2010, the path information of the lower body of the body model 1010 that is positioned in the imaging space 1036 from the initial position is simulated, so as to determine whether the implant identifier passes through the implant safety constraint region 1040.

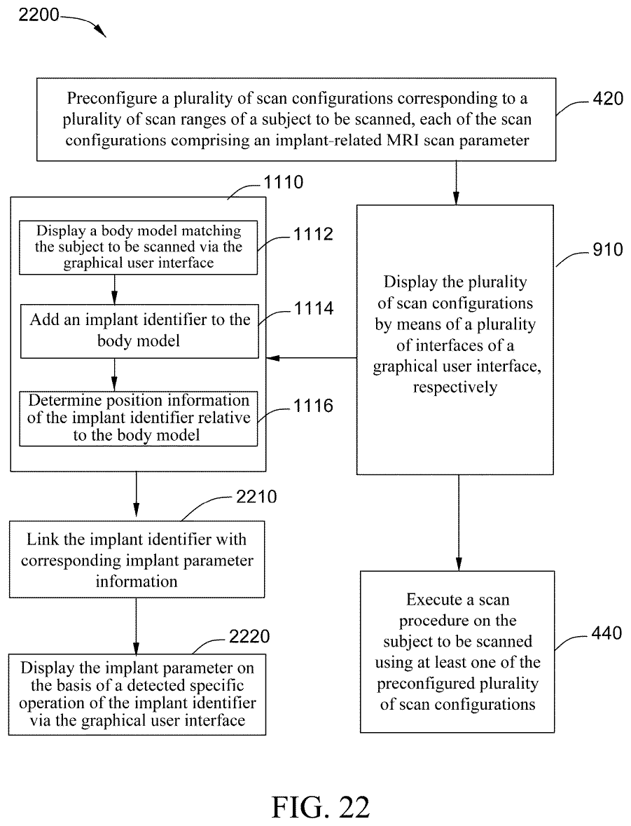

Referring to FIG. 22, FIG. 22 is a flowchart 2200 of an MRI scanning method according to another embodiment of the present invention. At step 2210, the implant identifier is linked to corresponding implant information which may be pre-acquired and stored in the patient information system 540 as previously described, and from which parameter information of the implant itself, e.g., description of implant type, manufacturer, parameter limits for the scanning system, etc., may be extracted; and the implant information may also include description documents, pictures, etc. In this way, a user opening and viewing a third-party database during an examination to confirm the implant parameters is avoided.

In step 2220, the implant information is displayed on the basis of a specific operation detected via the graphical user interface for the implant identifier. For example, when the user clicks the right mouse button at the implant identifier 1020 in the interface, the interface pops up the implant information, which may also include a link address that can link to a relevant document or picture.

In some embodiments, the displayed implant information may also be edited via the graphical user interface, such as modifying parameters or link addresses.

In the embodiments of the present invention, although it is described that a plurality of scan configurations related to an implant are preconfigured for different scan ranges, corresponding scan configurations may be configured for a plurality of different implants of a subject to be scanned, so that the corresponding scan configurations can be selected directly based on a predetermined scan procedure during a scanning process without interrupting the scan process for reconfiguration.

While the present invention has been described in detail with reference to specific embodiments, it would be understood by those skilled in the art that many modifications and variations can be made to the present invention. Therefore, it should be understood that the claims are intended to cover all such modifications and variations within the true spirit and scope of the present invention.

1. A magnetic resonance imaging (MRI) scanning method, comprising:
preconfiguring a plurality of scan configurations, the plurality of scan configurations corresponding to a plurality of scan ranges of a subject to be scanned, and each of the scan configurations comprising an implant-related MRI scan parameter; and
executing a scan procedure on the subject to be scanned using at least one of the preconfigured plurality of scan configurations.

2. The method according to claim 1, characterized in that the plurality of scan configurations comprise a first scan configuration and a second scan configuration, and the scan procedure comprises:
sequentially executing a first scan and a second scan on the basis of the first scan configuration and the second scan configuration, respectively.

3. The method according to claim 1, characterized by further comprising:
acquiring implant information of the subject to be scanned on the basis of a patient information system in communication with or integrated with an MRI scanning system; and
preconfiguring the plurality of scan configurations on the basis of the acquired implant information.

4. The method according to claim 1, characterized by further comprising: displaying the plurality of scan configurations by means of a plurality of interfaces of a graphical user interface, respectively.

5. The method according to claim 4, characterized in that preconfiguring a plurality of scan configurations comprises: configuring the plurality of scan configurations on the basis of a user input received via the graphical user interface.

6. The method according to claim 4, characterized by further comprising:
determining positioning information of an implant of the subject to be scanned in a body; and
generating safety prompt information in the plurality of scan configurations on the basis of the positioning information; and
displaying the safety prompt information in a corresponding plurality of scan configurations via the plurality of interfaces.

7. The method according to claim 6, characterized in that determining positioning information of an implant of the subject to be scanned in a body comprises:
displaying via the graphical user interface a body model matching the subject to be scanned;
adding an implant identifier to the body model; and
determining position information of the implant identifier relative to the body model.

8. The method according to claim 7, characterized in that adding an implant identifier to the body model comprises: adding the implant identifier to the body model in at least two different viewing angles, respectively.

9. The method according to claim 6, characterized in that determining positioning information of an implant of the subject to be scanned in a body comprises:
displaying via the graphical user interface a body model matching the subject to be scanned;
acquiring a medical image of the subject to be scanned on the basis of the patient information system in communication with the MRI scanning system;
matching the medical image with the body model;
determining the position information of the implant in the medical image; and
adding the implant identifier to a corresponding position of the body model on the basis of the determined position information.

10. The method according to claim 9, characterized in that matching the medical image with the body model comprises:
acquiring medical images in at least two different anatomical directions, respectively; and
matching the medical images in the at least two different anatomical directions with the body model in the at least two different viewing angles, respectively.

11. The method according to claim 7, characterized in that generating safety prompt information in the plurality of scan configurations on the basis of the positioning information comprises:
generating a simulation image based on the determined positioning information of the implant of the subject to be scanned in the body, the simulation image comprising a simulated model of the MRI scanning system, the body model with the implant identifier and an implant safety constraint region.

12. The method according to claim 11, characterized by further comprising: simulating path information of the body model entering an imaging space of the simulated model of the MRI scanning system on the basis of a scan range corresponding to a current scan configuration, so as to determine whether the implant identifier in the body model passes through the safety constraint region.

13. The method according to claim 7, characterized by further comprising:
acquiring body metric information of a subject to be tested; and
acquiring a body model matching the subject to be tested based on the body metric information.

14. The method according to claim 7, characterized by further comprising:
acquiring a three-dimensional image of the subject to be tested taken via a camera; and
generating a body model matching the subject to be scanned on the basis of the three-dimensional image.

15. The method according to claim 1, characterized in that preconfiguring a plurality of scan configurations comprises at least one of the following operations:
deleting at least one existing scan configuration;
editing at least one existing scan configuration; and
adding a new scan configuration.

16. The method according to claim 7, characterized by further comprising:
linking the implant identifier with corresponding implant parameter information; and
displaying the implant parameter on the basis of a specific operation of the implant identifier detected via the graphical user interface.

17. The method according to claim 16, characterized by further comprising: editing the displayed implant parameter via the graphical user interface.

18. An MRI scanning system, comprising:
a controller, used to execute the MRI scanning method according to claim 1; and
an MRI assembly, used to execute the scan procedure on the subject to be scanned on the basis of a control instruction of the controller.

19. An MRI scanning system, comprising:
an MRI assembly;
a controller; and
a graphical user interface interacting with the controller, the graphical user interface comprising a plurality of interfaces, the plurality of interfaces displaying a plurality of preset scan configurations, respectively, the plurality of scan configurations corresponding to a plurality of scan ranges of a subject to be scanned, and each of the scan configurations comprising an implant-related MRI scan parameter,
wherein the controller is used to control the MRI assembly to perform a scan procedure on the subject to be scanned using at least one of the preconfigured plurality of scan configurations.

20. The MRI scanning system according to claim 19, characterized in that each of the plurality of interfaces is also used to:
display a body model matching the subject to be scanned;
receive a user operation to add an implant identifier to the body model; and
display safety prompt information in a corresponding scan configuration, wherein the safety prompt information is generated on the basis of position information of the implant identifier in the body model.
